(19) United States
(12) Patent Application Publication
(10) Pub. No.: US 2025/0029723 A1
(43) Pub. Date: Jan. 23, 2025

(54) A SYSTEM AND METHOD FOR MEDICAL QUERIES

(71) Applicant: JIO PLATFORMS LIMITED, Ahmedabad (IN)

(72) Inventors: Chetan B. BHATT, Mumbai (IN); Shailesh KUMAR, Hyderabad (IN); Amrita Virendra VERMA, Mumbai (IN); Kalyan TADEPALLI, Mumbai (IN); Ritesh SHAH, Hyderabad (IN); Vamshi CHITTALA, Hyderabad (IN); Hareesh KUMAR, Hosur (IN); Neeraj YOGENDRA, Hyderabad (IN)

(73) Assignee: JIO PLATFORMS LIMITED, Ahmedabad (IN)

(21) Appl. No.: 18/713,072

(22) PCT Filed: Nov. 24, 2022
(86) PCT No.: PCT/IB2022/061370

§ 371 Date: May 23, 2024

(30) Foreign Application Priority Data
Nov. 24, 2021 (IN) 202121054099

(51) Int. Cl. G16H 50/20; G06F 16/242; G16H 10/60; G16H 50/30

(52) U.S. Cl. CPC G16H 50/20; G06F 16/243; G16H 10/60; G16H 50/30

(57) ABSTRACT

Present disclosure generally relates to computer-assisted medical diagnosis systems, particularly, to system and method for generating adaptive medical queries to determine disease symptoms of a user. System receives inputs from user, in response to queries corresponding to symptoms of disease. Further, system extracts contextual attributes corresponding to medical context, from inputs and identifies batch-wise candidate attributes from extracted contextual attributes, and sorts batch-wise candidate attributes corresponding to each of symptoms. Furthermore, system maps symptoms in sorted batch-wise candidate attributes to disease, by searching medical knowledge database, and calculates disease-symptom weighted score. Additionally, system filters symptoms based on age and gender of user, and sorts symptoms based on calculated disease-symptom weighed score and inter-dependency on other attributes. Furthermore, system generates subsequently, adaptive medical queries in human-understandable form, by predicting adaptive medical queries based on converting symptoms, attribute canonical names, and unique Identities (IDs), to determine disease of user.

### FIELD OF INVENTION

The embodiments of the present disclosure generally relate to a field of computer-assisted medical diagnosis systems. More particularly, the present disclosure relates generally to a system and a method for generating adaptive medical queries to determine one or more disease symptoms of a user.

### BACKGROUND OF THE INVENTION

The following description of related art is intended to provide background information pertaining to the field of the disclosure. This section may include certain aspects of the art that may be related to various features of the present disclosure. However, it should be appreciated that this section be used only to enhance the understanding of the reader with respect to the present disclosure, and not as admissions of prior art.

Generally, online symptom checkers are health tools that may help users diagnose a most likely disease(s) based on one or more symptoms provided by the user to the online symptom checkers. In a healthcare domain, a symptom-based triage may be known to be a critical and complex use-case scenario, due to various factors. The various factors include an impact and subjectivity of diagnoses, complex interactions between symptoms and diseases, knowledge engineering of vast medical information, and a reasoning-based inferencing required thereof. Further, the online symptom checkers may find a significant application in providing quick diagnoses to patients who do not have easy access to health facilities. Such online symptom checkers may also be referred by medical students to learn differential diagnosis procedures.

Conventionally, the online symptom-checkers or diagnostics-as-a-service products may use medical ontologies and web data coupled with knowledge-based or empirical-based approaches to achieve accurate disease predictions. The online symptom-checkers or diagnostics-as-a-service products may be available either as products or online web applications. Some of the online symptom-checkers may employ an interactive methodology in the form of question-answers in a chatbot-like setting, whereas some of the online symptom-checkers may predict diseases based only on initial symptoms inputted by a user. The key factors that interactive online symptom-checkers are typically evaluated using an accuracy of the differential diagnosis procedures, a coverage of the diseases and the symptoms, a correctness or relevance of questions asked to the user, an order in which the questions are asked, a number of questions that need to be answered to get an accurate diagnosis, and the like. Further, existing online symptom checkers may employ various automatic methods to elicit required medical data from the web. Often inconsistent or limited structured datasets on the web, may potentially lead to an inaccurate knowledge base at an outset. Furthermore, such data when used to train various machine learning or reasoning models tend to propagate the errors and eventually result in a less accurate diagnosis of the disease of the user.

Further, a conventional method provides a knowledge graph generating method, using a curated corpus of medical information, and receiving symptom inputs from the patient. Further, the knowledge graph generating method may weigh the extracted patient symptom and query a knowledge graph to generate a diagnosis graph as a subset of the knowledge graph. However, the conventional method may be limited to examining patient symptoms and demographics, and may not examine more details such as derived possible symptoms, symptom attributes, patient lifestyle, medical history, and the like. This leads to incomplete information about the patient's medical condition and in turn leads to inaccurate disease diagnosis of the disease of the user. The conventional method may not include a dialogue engine, which performs a two-way conversation between the user and a device used for diagnosing the disease of the user. Furthermore, another conventional method provides a computer software medical diagnosis system with which users can interact over the internet using a web browser to obtain a medical diagnosis or recommendation. However, the medical diagnosis system may not generate questions to examine the attributes of all symptoms present in the user, which could be incomplete information about the patient's medical condition and leads to inaccurate disease diagnosis of the user.

Therefore, there is a need in the art to provide systems and methods that can overcome the shortcomings of the existing prior art, by providing a system and a method for generating adaptive medical queries to determine the disease symptoms of a user.

### OBJECTS OF THE PRESENT DISCLOSURE

Some of the objects of the present disclosure, which at least one embodiment herein satisfy are as listed herein below.

An object of the present disclosure is to provide a system and a method for generating adaptive medical queries to determine the disease symptoms of a user.

Another object of the present disclosure is to provide a system and a method for facilitating predicting pertinent questions based on the various states as well as a sufficient number of questions with a view to converging a triage.

Another object of the present disclosure is to ensure the enforcement and priority of policy questions as mandated by experts.

Another object of the present disclosure is to provide a system and a method for enabling dependency between successive questions based on the answers to all previous questions.

Another object of the present disclosure is to provide a system and a method for covering questions based on contexts like age, gender, prior diseases, risk factors, lab tests, body organs, and body systems of the user.

Another object of the present disclosure is to provide benefits for healthcare to less privileged patients by collectively increasing the efficiency of conversation with a virtual doctor by asking appropriate questions which helps in providing quick diagnostics and reducing overall costs of disease diagnoses.

Another object of the present disclosure is to provide a system and a method for serving as a reference to medical students assisting them in carrying out a triage with the patient to pose the right questions at right time and diagnose the diseases accurately.

Another object of the present disclosure is to provide a system and a method for collecting consistent and adequate information from the patient which potentially leads to an accurate disease diagnosis and various machine learning models to learn the correct information.

### SUMMARY

This section is provided to introduce certain objects and aspects of the present invention in a simplified form that are further described below in the detailed description. This summary is not intended to identify the key features or the scope of the claimed subject matter.

In an aspect, the present disclosure provides a system for generating adaptive medical queries to determine the disease symptoms of a user. The system receives one or more inputs from a user, in response to the one or more queries corresponding to one or more symptoms associated with a disease of the user. Further, the system determines, if a previous query from the one or more queries is a symptomatic query, and determines if each of the one or more inputs from the user is an affirmative input. Furthermore, the system extracts a set of contextual attributes, an example of which, is the symptom Fever having a contextual attribute Range that spans from mild to very high. These attributes corresponding to a medical context are extracted from the one or more inputs received from the user, when the one or more queries is the symptomatic query, and each of the one or more inputs from the user is the affirmative input.

Additionally, the system identifies batch-wise candidate attributes from the extracted set of contextual attributes, and sorts the batch-wise candidate attributes corresponding to each of the one or more symptoms, in a display order. An example for the batch-wise candidate attribute is colour of the cough conditioned on the cough type. Further, the system maps the one or more symptoms in the sorted batch-wise candidate attributes to the disease, by searching a medical knowledge database. Furthermore, the system calculates a disease-symptom weighed score for the mapped one or more symptoms of the disease, by retrieving a symptom bucket corresponding to the disease in a knowledge graph. Additionally, the system filters the one or more symptoms based on an age and a gender of the user, and sorts the one or more symptoms based on the calculated disease-symptom weighed score and an inter-dependency on other attributes in the batch-wise candidate attributes of each symptom. Further, the system transmits the filtered and sorted one or more symptoms to a natural language generation layer for converting the one or more symptoms, attribute canonical names, and unique Identities (IDs) to a human-understandable form. Furthermore, the system generates subsequently, one or more adaptive medical queries in the human-understandable form, by predicting the one or more adaptive medical queries based on the converted one or more symptoms, the attribute canonical names, and the unique Identities (IDs), to determine the disease of the user.

Additionally, the system determines at least one of an insurance policy, an insurance premium, a health index, and a preventive measures guided by one or more health authorities, upon receiving a response from the user for the one or more adaptive medical queries. Further, the system generates the determined at least one of the insurance policy, the insurance premium, the health index, and the preventive measures guided by one or more health authorities.

In another aspect, the present disclosure provides a method for generating adaptive medical queries to determine disease symptoms of a user. The method includes receiving one or more inputs from a user, in response to one or more queries corresponding to one or more symptoms associated with a disease of the user. Further, the method includes determining, if a previous query from the one or more queries is a symptomatic query, and determining, if each of the one or more inputs from the user is an affirmative input. Furthermore, the method includes extracting a set of contextual attributes corresponding to a medical context, from the one or more inputs received from the user, when the one or more queries is the symptomatic query, and each of the one or more inputs from the user is the affirmative input. Additionally, the method includes identifying batch-wise candidate attributes from the extracted set of contextual attributes, and sorting the batch-wise candidate attributes corresponding to each of the one or more symptoms, in a display order. Further, the method includes mapping the one or more symptoms in the sorted batch-wise candidate attributes to the disease, by searching a medical knowledge database. Furthermore, the method includes calculating a disease-symptom weighed score for the mapped one or more symptoms to the disease, by retrieving a symptom bucket corresponding to the disease in a knowledge graph. Additionally, the method includes filtering the one or more symptoms based on an age and a gender of the user, and sorts the one or more symptoms based on the calculated disease-symptom weighed score and an inter-dependency on other attributes in the batch-wise candidate attributes of each symptom. Further, the method includes transmitting the filtered and sorted one or more symptoms to a natural language generation layer for converting the one or more symptoms, attribute canonical names, and unique Identities (IDs) to a human-understandable form. Furthermore, the method includes generating subsequently, one or more adaptive medical queries in the human-understandable form, by predicting the one or more adaptive medical queries based on the converted one or more symptoms, the attribute canonical names, and the unique Identities (IDs), to determine the disease of the user.

### BRIEF DESCRIPTION OF DRAWINGS

The accompanying drawings, which are incorporated herein, and constitute a part of this invention, illustrate exemplary embodiments of the disclosed methods and systems in which like reference numerals refer to the same parts throughout the different drawings. Components in the drawings are not necessarily to scale, emphasis instead being placed upon clearly illustrating the principles of the present invention. Some drawings may indicate the components using block diagrams and may not represent the internal circuitry of each component. It will be appreciated by those skilled in the art that invention of such drawings includes the invention of electrical components, electronic components, or circuitry commonly used to implement such components.

FIG. 1 illustrates an exemplary block diagram representation of a network architecture, in which or with which the system of the present disclosure can be implemented, in accordance with an embodiment of the present disclosure.

FIG. 2 illustrates an exemplary block representation of the proposed system for generating adaptive medical queries to determine disease symptoms of a user, in accordance with an embodiment of the present disclosure.

FIG. 3 illustrates an exemplary flow diagram representation depicting a method for synthesizing diagnosis utterance, in accordance with an embodiment of the present disclosure.

FIG. 4 illustrates an exemplary flow diagram representation depicting a method for predicting rule-based queries and reasoning-based queries, in accordance with an embodiment of the present disclosure.

FIG. 5 illustrates an exemplary flow diagram representation depicting a method for updating inputs from the user for the symptom queries, in accordance with an embodiment of the present disclosure.

FIG. 6 illustrates an exemplary flow diagram representation depicting a method for calculating symptom scores, in accordance with an embodiment of the present disclosure.

FIG. 7 illustrates an exemplary flow diagram representation depicting a method for generating adaptive medical queries to determine disease symptoms of a user, in accordance with an embodiment of the present disclosure.

FIG. 8 illustrates an exemplary computer system in which or with which embodiments of the present invention can be utilized in accordance with embodiments of the present disclosure.

The foregoing shall be more apparent from the following more detailed description of the invention.

### DETAILED DESCRIPTION OF INVENTION

In the following description, for the purposes of explanation, various specific details are set forth in order to provide a thorough understanding of embodiments of the present disclosure. It will be apparent, however, that embodiments of the present disclosure may be practiced without these specific details. Several features described hereafter can each be used independently of one another or with any combination of other features. An individual feature may not address all of the problems discussed above or might address only some of the problems discussed above. Some of the problems discussed above might not be fully addressed by any of the features described herein.

The ensuing description provides exemplary embodiments only, and is not intended to limit the scope, applicability, or configuration of the disclosure. Rather, the ensuing description of the exemplary embodiments will provide those skilled in the art with an enabling description for implementing an exemplary embodiment. It should be understood that various changes may be made in the function and arrangement of elements without departing from the spirit and scope of the invention as set forth.

Various embodiments of the present disclosure provide a system and a method for generating adaptive medical queries to determine disease symptoms of a user. The present disclosure provides a system and a method for facilitating predicting pertinent questions based on the various states as well as a sufficient number of questions with a view to converging a triage. The present disclosure ensures the enforcement and priority of policy questions as mandated by experts. The present disclosure provides a system and a method for enabling dependency between successive questions based on the answers to all previous questions. The present disclosure provides a system and a method for covering questions based on contexts like age, gender, prior diseases, risk factors, lab tests, body organs, and body systems of the user. The present disclosure provides benefits to healthcare to less privileged patients by collectively increasing the efficiency of conversation with a virtual doc by asking appropriate questions which help in providing quick diagnostics and reducing overall costs of disease diagnoses. The present disclosure provides a system and a method for serving as a reference to medical students assisting them in carrying out a triage with the patient to pose the right questions at right time and diagnose the diseases accurately. The present disclosure provides a system and a method for collecting consistent and adequate information from the patient which potentially leads to an accurate disease diagnosis and various machine learning models to learn the correct information.

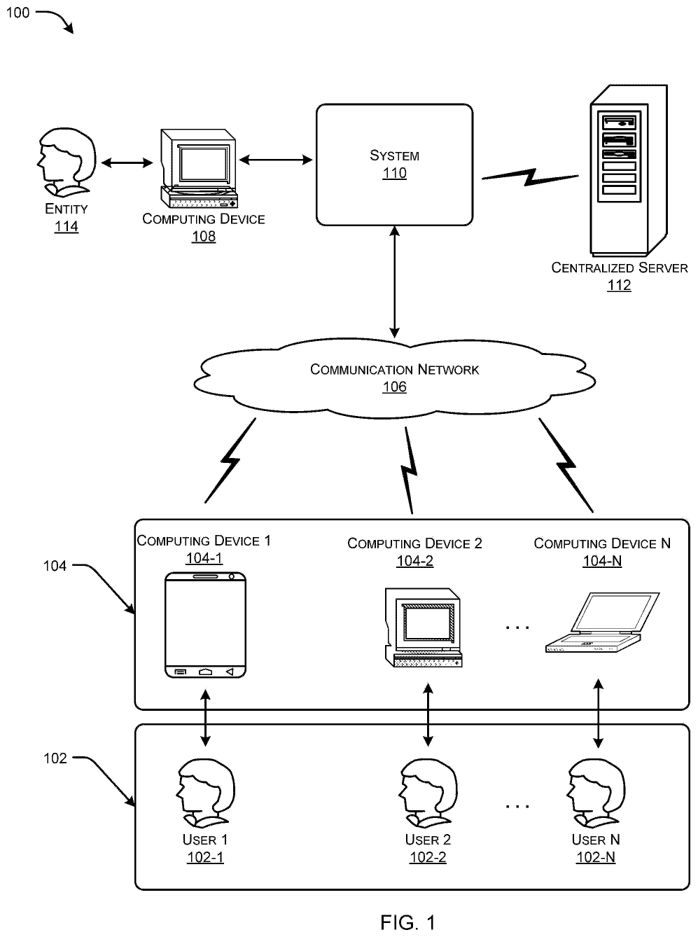

Referring to FIG. 1 that illustrates an exemplary network architecture for adaptive medical queries generating system (100) (also referred to as the network architecture (100)), in which or with which system (110) of the present disclosure can be implemented, in accordance with an embodiment of the present disclosure. As illustrated, the exemplary network architecture (100) may include the system (110) for generating adaptive medical queries to determine disease symptoms of a plurality of users (102-1, 102-2, . . . , 102-N) (individually referred to as the user (102), and collectively referred to as the users (102)) associated with one or more computing devices (104-1, 104-2, . . . , 104-N) (individually referred to as the computing device (104) and collectively referred to as the computing devices (104)). The users (102) may interact with the system (110) by using their respective computing device (104), wherein the computing device (104) and the system (110) may communicate with each other over a communication network (106).

Further, the system (110) may be further operatively coupled to a second computing device (108) associated with an entity (114). The entity (114) may include a company, an organization, a university, a lab facility, a company, a vendor, a manufacturing unit, a network operator, a business enterprise, a defence facility, a medical diagnosing facility, a hospital, an e-commerce platform, an internet service provider, an Over The Top (OTT) service provider, a media platform, or any other secured facility, and the like. In some implementations, the system (110) may also be associated with the second computing device (108). Further, the system (110) may also be communicatively coupled to the computing devices (104) via a communication network (106). In another implementation, the system (110) may also be associated with a centralized server (112). In yet another implementation the system (110) may be associated with the computing devices (104).

The system (110) may be coupled to the centralized server (112). The centralized server (112) may also be operatively coupled to the computing devices (104) and the second computing device (108) through the communication network (106). In some implementations, the system (110) may also be associated with the centralized server (112).

In an embodiment, the system (110) may receive one or more inputs from the user (102), in response to one or more queries corresponding to one or more symptoms associated with a disease of the user (102). In an embodiment, the one or more inputs comprise, but are not limited to, personal information of the user (102), and demographic information comprising the age, the gender of the user (102).

In an embodiment, the system (110) may determine, if a previous query from the one or more queries is a symptomatic query, and determine, if each of the one or more inputs from the user (102) is an affirmative input.

In an embodiment, the system (110) may extract a set of contextual attributes corresponding to a medical context, from the one or more inputs received from the user (102), when the one or more queries is the symptomatic query, and each of the one or more inputs from the user (102) is the affirmative input. In an embodiment, the medical context comprises, but is not limited to, attributes of the user (102), lifestyles of the user (102), demographics of the user (102), medical histories of the user (102), family histories of the user (102), prior diseases of the user (102), risk factors of the user (102), lab-tests of the user (102), problems in body organs of the user (102), body nature of the user (102), and the like.

In an embodiment, when the determined previous query from the one or more queries is not the symptomatic query, and the determined each of the one or more inputs from the user (102) is not the affirmative input, the system (110) may further receive one or more symptoms of the disease as an input to one or more queries which are reasoning-based queries, from the user (102). In an embodiment, the system (110) may determine top ‘n’ disease scores with a highest probability, based on the received one or more symptoms of the disease, and the age and the gender of the user (102). In an embodiment, the system (110) may determine top ‘k’ disease scores, by calculating a heuristic score using the received one or more symptoms. In an embodiment, the system (110) may determine candidate symptoms of the determined top ‘k’ disease scores, and compute candidate symptom scores based on the symptom bucket, and pre-defined disease scores. In an embodiment, the system (110) may analyze, if the top ‘n’ disease scores are greater or equal to the top ‘k’ disease scores with respect to the computed candidate symptom scores. In an embodiment, the system (110) may map the candidate symptoms to the disease, by searching the medical knowledge database, when the top ‘n’ disease scores are greater or equal to the top ‘k’ disease scores. In an embodiment, the system (110) may calculate the disease-symptom weighed score for the mapped candidate symptoms to the disease, by retrieving a symptom bucket corresponding to the disease in a knowledge graph. In an embodiment, the system (110) may filter the one or more symptoms based on the age and the gender of the user (102), and sort the one or more symptoms based on the calculated disease-symptom weighed score and the pre-defined disease scores. In an embodiment, the system (110) may transmit the filtered and sorted candidate symptoms to the natural language generation layer for converting the candidate symptoms to the human-understandable form.

In an embodiment, the system (110) may identify batch-wise candidate attributes from the extracted set of contextual attributes, and sort the batch-wise candidate attributes corresponding to each of the one or more symptoms, in a display order. An example for the batch-wise candidate attribute may be a colour of a cough conditioned on a cough type, and similar other examples.

In an embodiment, the system (110) may map the one or more symptoms in the sorted batch-wise candidate attributes to the disease, by searching a medical knowledge database.

In an embodiment, the system (110) may calculate a disease-symptom weighed score for the mapped one or more symptoms of the disease, by retrieving a symptom bucket corresponding to the disease in a knowledge graph. In an embodiment, for calculating the disease-symptom-weighed score, the system (110) may iterate over all diseases, to sum the disease-symptom-weighted score for each disease. Further, the system (110) may determine a probability score of each disease, for the summed-up disease-symptom weighted score. Furthermore, the system (110) may determine the sum of the probability score of all the diseases. Further, the system (110) may calculate a normalized disease score from the probability score of each disease and the sum of the probability score of all the diseases. Furthermore, the system (110) may calculate a symptom score of each disease, based on the disease-symptom weightage score and the normalized disease score. Furthermore, the system (110) may update the symptom score for each disease, and update a final symptom score, by summing the symptom score of each disease.

In an embodiment, the system (110) may filter the one or more symptoms based on an age and a gender of the user (102), and sort the one or more symptoms based on the calculated disease-symptom weighed score and an inter-dependency on other attributes in the batch-wise candidate attributes of each symptom.

In an embodiment, the system (110) may transmit the filtered and sorted one or more symptoms to a natural language generation layer for converting the one or more symptoms, attribute canonical names, and unique Identities (IDs) to a human-understandable form.

In an embodiment, the system (110) may generate subsequently, one or more adaptive medical queries in the human-understandable form, by predicting the one or more adaptive medical queries based on the converted one or more symptoms, the attribute canonical names, and the unique Identities (IDs), to determine the disease of the user (102). In an embodiment, the generated one or more adaptive medical queries in the human-understandable form are rule-based queries. In an embodiment, the one or more adaptive medical queries are predicted by analyzing previous inputs from the user (102), based on, but are not limited to, previous inputs corresponding to the demographic of the user (102), previous inputs corresponding to the symptoms of the user (102), previous inputs corresponding to the attributes, and the like.

In an embodiment, the system (110) may determine at least one of an insurance policy, an insurance premium, a health index, and a preventive measures guided by one or more health authorities, upon receiving a response from the user for the one or more adaptive medical queries. Further, the system (110) may generate the determined at least one of the insurance policy, the insurance premium, the health index, and the preventive measures guided by one or more health authorities. For example, the system (110) may retrieve data corresponding to at least one of an insurance policy, an insurance premium, a health index, and a preventive measures guided by one or more health authorities, from one or more databases/regulatory repositories. Further, the system (110) may map the data from the one or more databases/regulatory repositories to the responses from the user for the one or more adaptive medical queries, to determine the at least one of the insurance policy, the insurance premium, the health index, and the preventive measures guided by one or more health authorities.

In an embodiment, the system (110) may be a System on Chip (SoC) system but not limited to the like. In another embodiment, an onsite data capture, storage, matching, processing, decision-making, and actuation logic may be coded using Micro-Services Architecture (MSA) but not limited to it. A plurality of microservices may be containerized and may be event-based to support portability.

In an embodiment, the network architecture (100) may be modular and flexible to accommodate any kind of changes in the system (110) as proximate processing may be acquired towards generating adaptive medical queries to determine the disease symptoms of a user. The system (110) configuration details can be modified on the fly.

In an embodiment, the system (110) may be remotely monitored and the data, application, and physical security of the system (110) may be fully ensured. In an embodiment, the data may get collected meticulously and deposited in a cloud-based data lake to be processed to extract actionable insights. Therefore, the aspect of predictive maintenance can be accomplished.

In an exemplary embodiment, the communication network (106) may include, by way of example but not limitation, at least a portion of one or more networks having one or more nodes that transmit, receive, forward, generate, buffer, store, route, switch, process, or a combination thereof, etc. one or more messages, packets, signals, waves, voltage or current levels, some combination thereof, or so forth. A network may include, by way of example but not limitation, one or more of: a wireless network, a wired network, an internet, an intranet, a public network, a private network, a packet-switched network, a circuit-switched network, an ad hoc network, an infrastructure network, a Public-Switched Telephone Network (PSTN), a cable network, a cellular network, a satellite network, a fiber-optic network, a Bluetooth, a Zigbee, a Message Queuing Telemetry Transport (MQTT) broker cloud, Local Area Network (LAN), Wide Area Network (WAN), some combination thereof, and the like. Further, the communication network (106) can either be a dedicated network or a shared network. The shared network can represent an association of the different types of networks that can use a variety of protocols, for example, Hypertext Transfer Protocol (HTTP), Transmission Control Protocol/Internet Protocol (TCP/IP), Wireless Application Protocol (WAP), and the like. In an exemplary embodiment, the network 104 can be an HC-05 Bluetooth module which is an easy-to-use Bluetooth Serial Port Protocol (SPP) module, designed for transparent wireless serial connection setup.

In another exemplary embodiment, the centralized server (112) may include or comprise, by way of example but not limitation, one or more of: a stand-alone server, a server blade, a server rack, a bank of servers, a server farm, hardware supporting a part of a cloud service or system, a home server, hardware running a virtualized server, one or more processors executing code to function as a server, one or more machines performing server-side functionality as described herein, at least a portion of any of the above, some combination thereof.

In an embodiment, the computing devices (104), and the computing devices (108) may communicate with the system (110) via a set of executable instructions residing on any operating system, including but not limited to, Android™, iOS™, Kai OS™, and the like. In an embodiment, the computing devices (104), and the second computing device (108) may include, but are not limited to, any electrical, electronic, electro-mechanical or an equipment or a combination of one or more of the above devices such as healthcare and hospital assets, mobile phones, smartphones, Virtual Reality (VR) devices, Augmented Reality (AR) devices, laptops, general-purpose computers, desktops, personal digital assistants, tablet computers, mainframe computers, smart Televisions (TVs), smart set-top boxes, or any other computing device. The computing device may include one or more in-built or externally coupled accessories including, but not limited to, a visual aid device such as a camera, an audio aid, a microphone, a keyboard, input devices for receiving input from a user such as a touchpad, touch-enabled screen, electronic pen, receiving devices for receiving any audio or visual signal in any range of frequencies and transmitting devices that can transmit any audio or visual signal in any range of frequencies. It may be appreciated that the computing devices (104), and the second computing device (108) may not be restricted to the mentioned devices and various other devices may be used. A smart computing device may be one of the appropriate systems for storing data and other private/sensitive information.

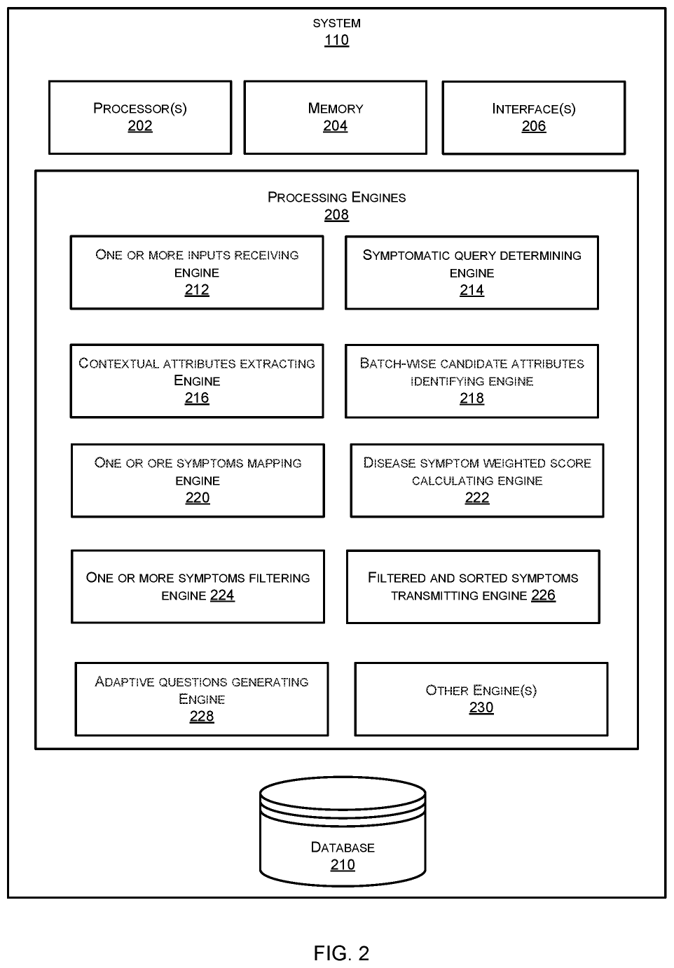

FIG. 2 illustrates an exemplary block representation of the proposed system (110) for generating adaptive medical queries to determine disease symptoms of the user (102), in accordance with an embodiment of the present disclosure.

In an aspect, the system (110) may include one or more processor(s) (202). The one or more processor(s) (202) may be implemented as one or more microprocessors, microcomputers, microcontrollers, edge or fog microcontrollers, digital signal processors, central processing units, logic circuitries, and/or any devices that process data based on operational instructions. Among other capabilities, one or more processor(s) (202) may be configured to fetch and execute computer-readable instructions stored in a memory (204) of the system (110). The memory (204) may be configured to store one or more computer-readable instructions or routines in a non-transitory computer-readable storage medium, which may be fetched and executed to create or share data packets over a network service. The memory (204) may comprise any non-transitory storage device including, for example, volatile memory such as a Random-Access Memory (RAM), or a Non-Volatile Memory (NVM) such as an Erasable Programmable Read-Only Memory (EPROM), flash memory, and the like.

In an embodiment, the system (110) may include an interface(s) 206. The interface(s) (206) may comprise a variety of interfaces, for example, interfaces for data input and output devices, referred to as I/O devices, storage devices, and the like. The interface(s) (206) may facilitate communication of the system (110). The interface(s) (206) may also provide a communication pathway for one or more components of the system (110). Examples of such components include, but are not limited to, processing unit/engine(s) (208) and a database (210).

The processing unit/engine(s) (208) may be implemented as a combination of hardware and programming (for example, programmable instructions) to implement one or more functionalities of the processing engine(s) (208). In examples described herein, such combinations of hardware and programming may be implemented in several different ways. For example, the programming for the processing engine(s) (208) may be processor-executable instructions stored on a non-transitory machine-readable storage medium and the hardware for the processing engine(s) (208) may comprise a processing resource (for example, one or more processors), to execute such instructions. In the present examples, the machine-readable storage medium may store instructions that, when executed by the processing resource, implement the processing engine(s) (208). In such examples, the system (110) may comprise the machine-readable storage medium storing the instructions and the processing resource to execute the instructions, or the machine-readable storage medium may be separate but accessible to the system (110) and the processing resource. In other examples, the processing engine(s) (208) may be implemented by electronic circuitry.

The processing engine (208) may include one or more engines selected from any of a one or more inputs receiving engine (212), a symptomatic query determining engine (214), a contextual attributes extracting engine (216), a batch-wise candidates attributes identifying engine (218), a one or more symptoms mapping engine (220), a disease symptom weighted score calculating engine (222), a one or more symptoms filtering engine (224), a filtered and sorted symptoms transmitting engine (226), an adaptive questions generating engine (228), and other engines/units (222). The processing engine (208) may further edge-based micro service event processing but is not limited to the like.

In an embodiment, the one or more inputs receiving engine (212) may receive one or more inputs from the user (102), in response to one or more queries corresponding to one or more symptoms associated with a disease of the user (102). In an embodiment, the one or more inputs comprise, but are not limited to, personal information of the user (102), and demographic information comprising the age, the gender of the user (102).

In an embodiment, the symptomatic query determining engine (214) may determine, if a previous query from the one or more queries is a symptomatic query, and determine, if each of the one or more inputs from the user (102) is an affirmative input.

In an embodiment, the contextual attributes extracting engine (216) may extract a set of contextual attributes corresponding to a medical context, from the one or more inputs received from the user (102), when the one or more queries is the symptomatic query, and each of the one or more inputs from the user (102) is the affirmative input. In an embodiment, the medical context comprises, but is not limited to, attributes of the user (102), lifestyles of the user (102), demographics of the user (102), medical histories of the user (102), family histories of the user (102), prior diseases of the user (102), risk factors of the user (102), lab-tests of the user (102), problems in body organs of the user (102), body nature of the user (102), and the like.

In an embodiment, when the determined previous query from the one or more queries is not the symptomatic query, and the determined each of the one or more inputs from the user (102) is not the affirmative input, the system (110) may further receive one or more symptoms of the disease as an input to one or more queries which are reasoning-based queries, from the user (102). In an embodiment, the system (110) may determine top ‘n’ disease scores with a highest probability, based on the received one or more symptoms of the disease, and the age and the gender of the user (102). In an embodiment, the system (110) may determine top ‘k’ disease scores, by calculating a heuristic score using the received one or more symptoms. In an embodiment, the system (110) may determine candidate symptoms of the determine top ‘k’ disease scores, and compute candidate symptom scores based on the symptom bucket, and pre-defined disease score. In an embodiment, the system (110) may analyze, if the top ‘n’ disease scores are greater or equal to the top ‘k’ disease scores with respect to the computed candidate symptom scores. In an embodiment, the system (110) may map the candidate symptoms to the disease, by searching the medical knowledge database, when the top ‘n’ disease scores are greater or equal to the top ‘k’ disease scores. In an embodiment, the system (110) may calculate the disease-symptom weighed score for the mapped candidate symptoms to the disease, by retrieving a symptom bucket corresponding to the disease in a knowledge graph. In an embodiment, the system (110) may filter the one or more symptoms based on the age and the gender of the user (102), and sort the one or more symptoms based on the calculated disease-symptom weighed score and the pre-defined deceased scores. In an embodiment, the system (110) may transmit the filtered and sorted candidate symptoms to the natural language generation layer for converting the candidate symptoms to the human-understandable form.

In an embodiment, the batch-wise candidates attributes identifying engine (218) may identify batch-wise candidate attributes from the extracted set of contextual attributes, and sort the batch-wise candidate attributes corresponding to each of the one or more symptoms, in a display order. An example for the batch-wise candidate attribute may be a colour of a cough conditioned on a cough type, and similar other examples.

In an embodiment, the one or more symptoms mapping engine (220) may map the one or more symptoms in the sorted batch-wise candidate attributes to the disease, by searching a medical knowledge database.

In an embodiment, the disease symptom-weighted score calculating engine (222) may calculate a disease-symptom-weighed score for the mapped one or more symptoms to the disease, by retrieving a symptom bucket corresponding to the disease in a knowledge graph. In an embodiment, for calculating the disease-symptom-weighed score, the system (110) may iterate over all diseases, to sum the disease-symptom-weighted score for each disease. Further, the system (110) may determine a probability score of each disease, for the summed-up disease-symptom weighted score. Furthermore, the system (110) may determine the sum of the probability score of all the diseases. Further, the system (110) may calculate a normalized disease score from the probability score of each disease and the sum of the probability score of all the diseases. Furthermore, the system (110) may calculate a symptom score of each disease, based on the disease-symptom weightage score and the normalized disease score. Furthermore, the system (110) may update the symptom score for each disease, and update a final symptom score, by summing the symptom score of each disease.

In an embodiment, the one or more symptoms filtering engine (224) may filter the one or more symptoms based on an age and a gender of the user (102), and sort the one or more symptoms based on the calculated disease-symptom weighed score and an inter-dependency on other attributes in the batch-wise candidate attributes of each symptom.

In an embodiment, the filtered and sorted symptoms transmitting engine (226) may transmit the filtered and sorted one or more symptoms to a natural language generation layer for converting the one or more symptoms, attribute canonical names, and unique Identities (IDs) to a human-understandable form.

In an embodiment, the adaptive questions generating engine (228) may generate subsequently, one or more adaptive medical queries in the human-understandable form, by predicting the one or more adaptive medical queries based on the converted one or more symptoms, the attribute canonical names, and the unique Identities (IDs), to determine the disease of the user (102). In an embodiment, the generated one or more adaptive medical queries in the human-understandable form are rule-based queries. In an embodiment, the one or more adaptive medical queries are predicted by analyzing previous inputs from the user (102), based on, but are not limited to, previous inputs corresponding to the demographic of the user (102), previous inputs corresponding to the symptoms of the user (102), previous inputs corresponding to the attributes, and the like.

In an embodiment, the system (110) may determine at least one of an insurance policy, an insurance premium, a health index, and a preventive measures guided by one or more health authorities, upon receiving a response from the user for the one or more adaptive medical queries. Further, the system (110) may generate the determined at least one of the insurance policy, the insurance premium, the health index, and the preventive measures guided by one or more health authorities.

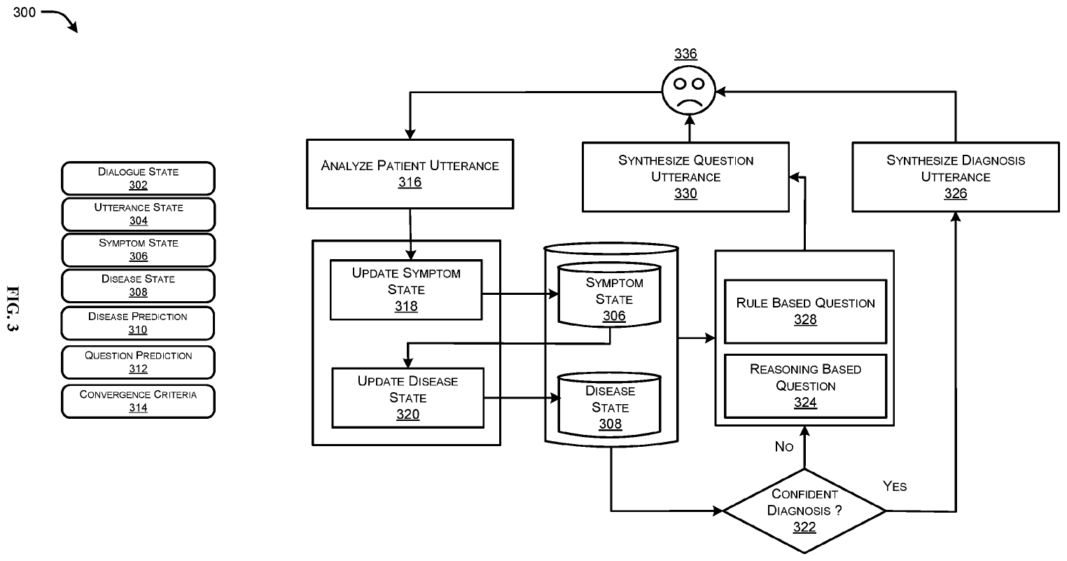

FIG. 3 illustrates an exemplary flow diagram representation depicting a method (300) for synthesizing diagnosis utterances, in accordance with an embodiment of the present disclosure.

In an exemplary embodiment, the system (110) may include a dialogue state module (302) that may be configured to orchestrate a conversation between the user (102) and the system (110) (e.g., Artificial Intelligence (AI) doctor), by keeping the state of the conversation so far including the questions asked by the system (110) and the answers given by the user (102) to the questions. In an embodiment, the system (110) may include an utterance state module (304). Exchanges that occur between the user (102) and the system (110), such as a response from the user (102) and a question from the system (110). The system (110) may include a symptom state module (306) that may include representing symptom-state in terms of presence/absence and attributes. The system (110) may further include a disease state module (308) that may determine the probability of whether a disease is present or not present for given symptoms. The system (110) may include a disease prediction module (310) that may determine given the current set of symptoms what is the probability of each disease. The system (110) may include a question prediction module (312) that may predict, given the symptom and disease state what is the next best question to ask. Further, the system (110) may include a convergence criteria module (314).

At step (316), the method (300) includes analyzing, by the system (110) a patient (i.e., user (102) utterance.

At step (318), the method (300) includes updating, by the system (110) a symptom state and sending it to a symptom state module (306).

At step (320), the method (300) includes outputting, by the system (110), from the symptom state module (306) to update a disease state and store it in the disease state module (308).

At step (322), the method (300) includes determining, by the system (110), if the diagnosis of the disease is confident.

At step (324), the method (300) includes providing, by the system (110), if the diagnosis is not confident, a reasoning (332) based question to the user (102).

At step (326), the method (300) includes, synthesizing, by the system (11), if the diagnosis is confident, a diagnosis utterance.

At step (328), the method (300) includes, outputting, by the system (110), from the symptom state module (306) and the disease state module (308) to a rule-based question module.

At step (330), the method (300) includes, synthesizing, by the system (110), a question utterance received from the rule-based question module.

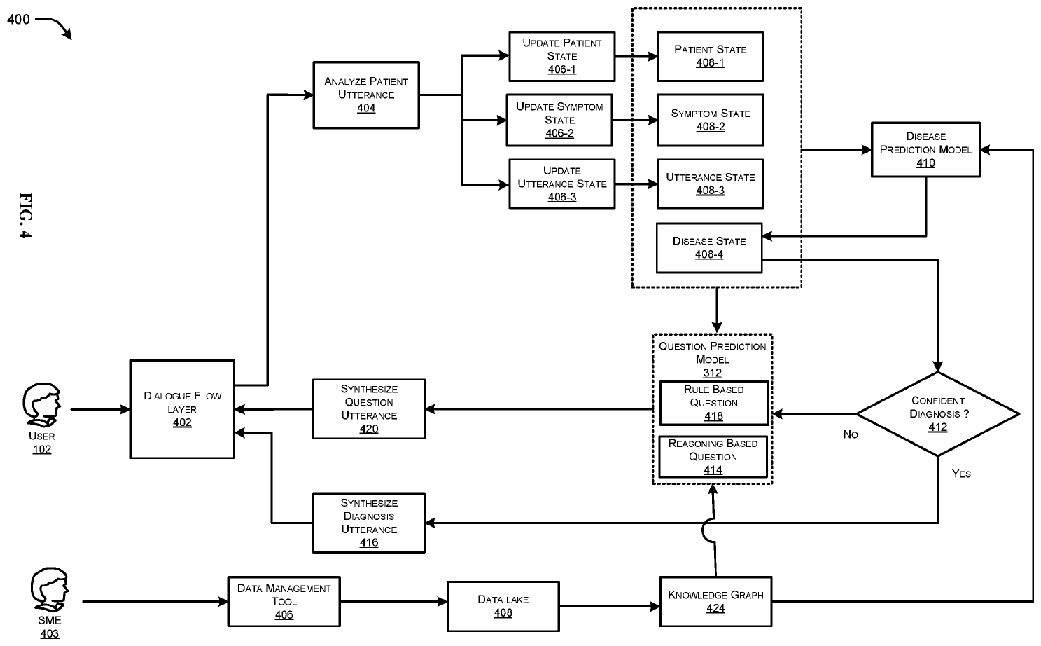

FIG. 4 illustrates an exemplary flow diagram representation depicting a method (400) for predicting rule-based queries and reasoning-based queries, in accordance with an embodiment of the present disclosure.

At step (402) the method (400) includes receiving, an input from the user (102) by a dialogue flow layer.

At step (404), the method (400) includes analyzing, by the system (100), the input (i.e., utterance) from the user (102).

At steps (406-1) (406-2) and (406-3), the method (400) includes updating, by the system (110), a patient state, a symptom state, and an utterance of the user (102).

At steps (408-1) (408-2) and (408-3), the method (400) includes sending, by the system (110), the updated patient state, a symptom state, and an utterance of the user (102) to a patient state module (422), the symptom state module (306), the utterance state module (304), and the disease state module (308).

At step (410), the method (400) includes outputting, by the system (110), the output from the patient state module (422), the symptom state module (306), the utterance state module (304), and the disease state module (308), to a disease prediction model.

At step (408-4), the method (400) includes, outputting, by the system (110), the output from the disease prediction model to the disease state module (308).

At step (412), the method (400) includes determining, by the system (110), if the diagnosis is confident, based on receiving the output from the disease state module (308).

At step (414), the method (400) includes, providing, by the system (110), if the diagnosis is not confident, reasoning-based question.

At step (416), the method (400) includes, synthesizing, by the system (110), if the diagnosis is confident, a diagnosis utterance.

At step (418), the method (400) includes, providing, by the system (110), an output from the symptom state module (306) and the disease state module (308) to a rule-based question module.

At step (420), the method (400) includes synthesizing, by the system (110) a question utterance. The output of the synthesized question utterance may be provided to the user (102).

The input may be received from a Subject Matter Expert (SME) user (403), which is then sent to a management tool (406), to a data lake (408), to a knowledge graph (424), and the question prediction model (312).

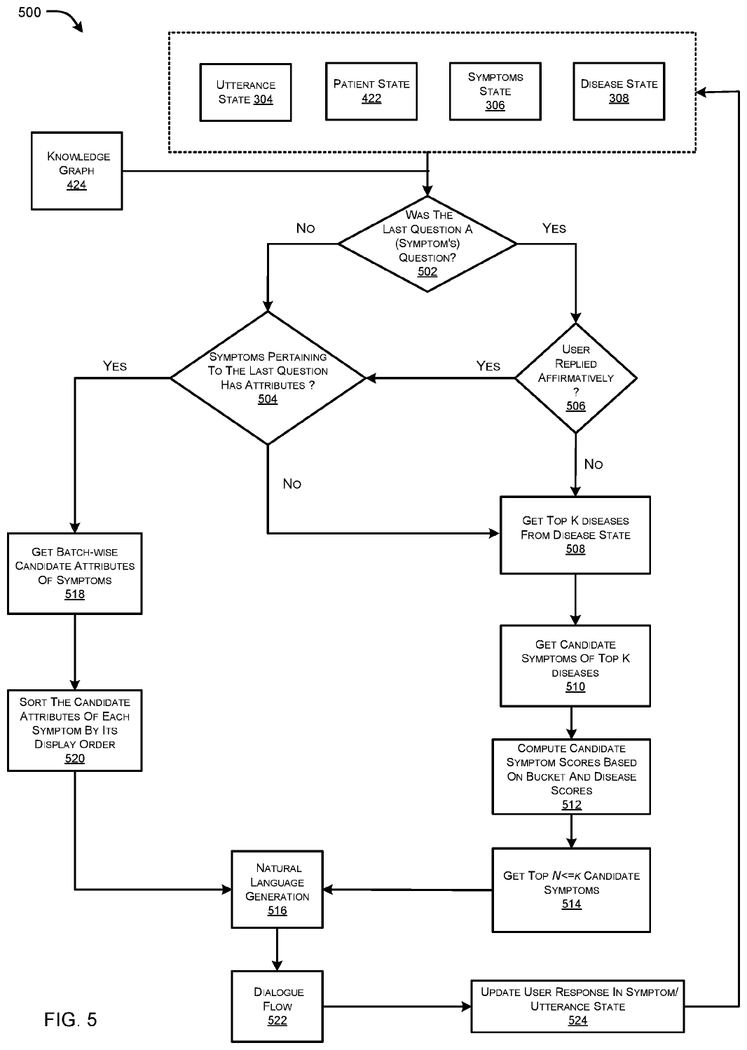

FIG. 5 illustrates an exemplary flow diagram representation depicting a method (500) for updating inputs from the user for the symptom queries, in accordance with an embodiment of the present disclosure.

At step (502), the method (500) includes checking, by the system (110), the data from a knowledge graph (424), and the data from any or a combination of the utterance state module (304), the patient state module (422), the symptom state module (306), the disease state module (308), if a last question was a symptom question.

At step (504), the method (500) includes determining, by the system (110), if the symptoms pertaining to a last question include attributes, when the last question is not the symptom question.

At step (506), the method (500) includes determining, by the system (110), if the user (102) has replied affirmatively to the last question, when the last question is the symptom question. If yes, at step (504) determining, by the system (110), if the symptoms pertaining to a last question include attributes.

At step (508), the method (500) includes determining, by the system (110), a top ‘k’ diseases with the highest probability from the disease state module (308), using the previous utterance question, patient's response for the utterance from the utterance state module (304), the user answered symptoms from the symptom state module (306), the patient's demographics such as age and gender from the patient state module (422), when the user has not affirmatively replied and symptoms pertaining to last questions do not include attributes.

At step (510), the method (500) includes determining, by the system (110), candidate symptoms of top k diseases.

At step (512), the method (500) includes computing, by the system (110), candidate symptom score, based on bucket and disease score. The method (500) may include a symptom scores calculator for question prediction using disease state (reasoning-based). The method (500) includes finding the highly likely diseases based on the heuristic score, filtering the symptoms mapped to likely diseases based on age and gender, and sorting the symptoms based on scores derived from the weights and disease score to reassure the likely diseases. The symptom score may be calculated as shown in equation 1 below:

$$σ ( s ) = {\sum }_{d ∈ D ( s )} SW ( s , d ) \times ( \frac{ϕ {( d )}^{β}}{{\sum }_{d ∈ D ( s )} ϕ {( d )}^{β}} ) \qquad Equation 1$$

σ(s)=Next Symptom Score
   In the equation 1, the variables
D(s)
   SW(s,d)
ϕ(d)
β
   The symptom score function gives higher weightages to those symptoms that are highly associated with diseases that are more likely to be present in the patient given the conversation so far.

At step (516), the method (500) includes, sending by the system (110), the filtered and sorted symptoms to the natural language generation layer for conversion from Identities (IDs) to patient understandable form.

At step (518), the method (500) includes determining, by the system (110), batch-wise candidate attributes of symptoms, when the symptoms pertaining to last question include attributes.

At step (520), the method (500) includes sorting, by the system (110), the determined batch-wise candidate attributes of symptoms in a display order, and at step (516), sending to the sorted batch-wise candidate attributes of symptoms to the natural language generation layer.

The system (110) may understand the previous utterances to decide the next utterance and then decision making based on previous demographic utterance response, provide decision making based on previous symptom utterance response, decision making based on previous attribute response. Additionally, in a rule-based method, sort all the attribute questions of the affirmative symptom(s) answered by the patient in the order of display or other preference provided by the experts, filter the attribute questions based on the age and gender of the patient, sort the attribute questions based on the inter-dependency on other attributes of each symptom, filter the attribute values based on age and gender for every attribute question of affirmative symptom(s) and send the filtered and sorted attributes to the Natural language generation layer for conversion from IDs to patient understandable form. A Natural language generation module may convert the symptoms and attribute canonical names and unique IDs into the format of a question that is readable by the patient and update the utterance state with the next set of symptoms and attributes IDs to mark the questions as asked to the patient.

At step (522), the method (500) includes generating, by the system (110), a question as a dialogue flow from the output of the Natural language generation layer.

At step (524), the method (500) includes updating, by the system (110), a user response to the question in the symptom state module (306) or the utterance state module (304).

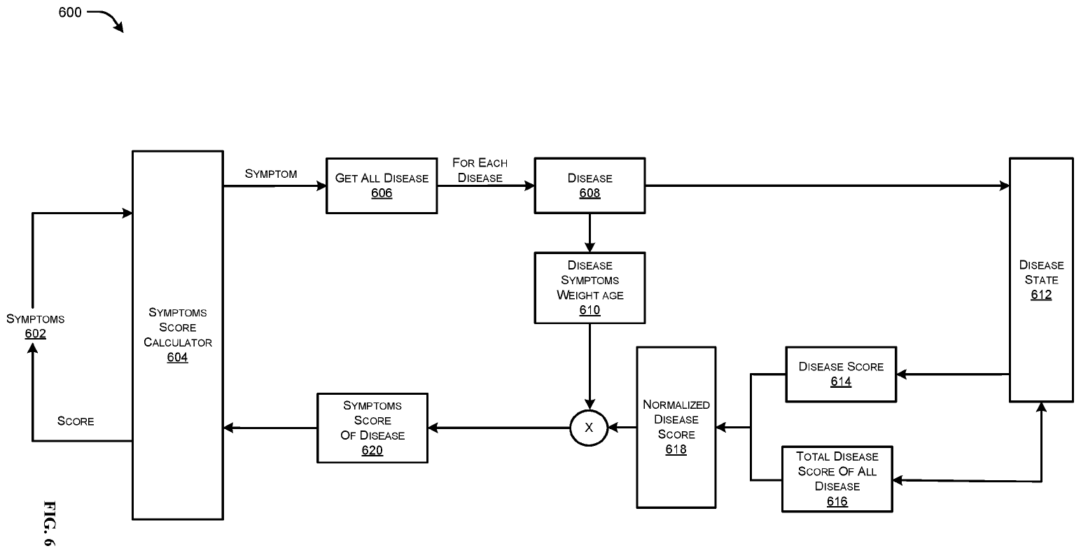

FIG. 6 illustrates an exemplary flow diagram representation depicting a method for calculating symptom scores, in accordance with an embodiment of the present disclosure.

At step (602) the method (600) includes providing, by the system (110), symptoms to a symptoms score calculator.

At step (604) the method (600) includes calculating, by the system (110), scores for the symptoms.

At step (606) the method (600) includes obtaining, by the system (110), all the diseases that are mapped to the given symptoms.

At step (608) the method (600) includes, by the system (110), iterating over all diseases, to sum up, the disease-specific symptom score for each disease.

At step (610) the method (600) includes calculating, by the system (110), the disease-symptom weightage, by fetching the symptom bucket in the disease from the knowledge graph.

At steps (612) and (614), the method (600) includes getting, by the system (110), the probability score of the disease from the disease state module (308).

At step (616), the method (600) includes, calculating, by the system (110), the sum of the probabilities of all the diseases (620) from the disease state module (308).

At step (618), the method (600) includes, calculating, by the system (110), a normalized disease score (616).

At step (620), the method (600) includes, calculating, by the system (110), using the disease-symptom weightage and normalized disease score, and a multiplicator (605), the symptom score of the disease.

The method (600) may further include the steps of updating the symptom score in the symptom score calculator for each symptom and updating the final symptom score by summing up the symptom score of each disease.

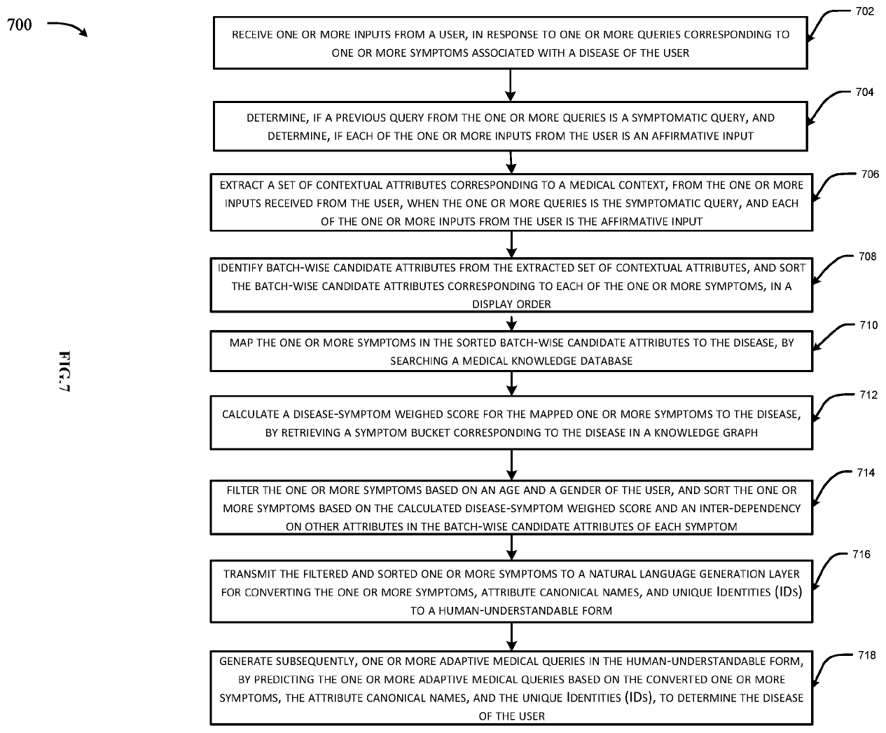

FIG. 7 illustrates an exemplary flow diagram representation depicting a method (700) for generating adaptive medical queries to determine disease symptoms of a user, in accordance with an embodiment of the present disclosure.

At block (702), the method (700) includes receiving, by a processor (202) associated with a system (110), one or more inputs from a user (102), in response to one or more queries corresponding to one or more symptoms associated with a disease of the user (102).

At block (704), the method (700) includes determining, by the processor (202), if a previous query from the one or more queries is a symptomatic query, and determining, if each of the one or more inputs from the user (102) is an affirmative input.

At block (706), the method (700) includes extracting, by the processor (202), a set of contextual attributes corresponding to a medical context, from the one or more inputs received from the user (102), when the one or more queries is the symptomatic query, and each of the one or more inputs from the user (102) is the affirmative input.

At block (708), the method (700) includes identifying, by the processor (202), batch-wise candidate attributes from the extracted set of contextual attributes, and sorting the batch-wise candidate attributes corresponding to each of the one or more symptoms, in a display order.

At block (710), the method (700) includes mapping, by the processor (202), the one or more symptoms in the sorted batch-wise candidate attributes to the disease, by searching a medical knowledge database.

At block (712), the method (700) includes calculating, by the processor (202), a disease-symptom weighed score for the mapped one or more symptoms to the disease, by retrieving a symptom bucket corresponding to the disease in a knowledge graph.

At block (714), the method (700) includes filtering, by the processor (202), the one or more symptoms based on an age and a gender of the user (102), and sorting the one or more symptoms based on the calculated disease-symptom weighed score and an inter-dependency on other attributes in the batch-wise candidate attributes of each symptom.

At block (716), the method (700) includes transmitting, by the processor (202), the filtered and sorted one or more symptoms to a natural language generation layer for converting the one or more symptoms, attribute canonical names, and unique Identities (IDs) to a human-understandable form.

At block (718), the method (700) includes generating, by the processor (202), subsequently, one or more adaptive medical queries in the human-understandable form, by predicting the one or more adaptive medical queries based on the converted one or more symptoms, the attribute canonical names, and the unique Identities (IDs), to determine the disease of the user (102).

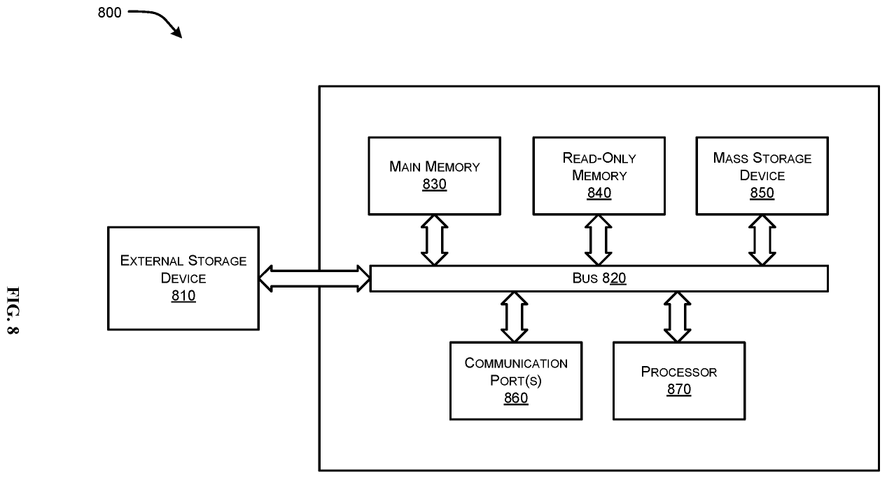

FIG. 8 illustrates an exemplary computer system in which or with which embodiments of the present invention can be utilized in accordance with embodiments of the present disclosure. As shown in FIG. 8, computer system 800 can include an external storage device 810, a bus 820, a main memory 830, a read-only memory 840, a mass storage device 870, a communication port 860, and a processor 870. A person skilled in the art will appreciate that the computer system may include more than one processor and communication ports. Examples of processor 870 include, but are not limited to, an Intel® Itanium® or Itanium 2 processor(s), or AMD® Opteron® or Athlon MP® processor(s), Motorola® lines of processors, FortiSOC™ system on chip processors or other future processors. Processor 870 may include various modules associated with embodiments of the present invention. Communication port 860 can be any of an RS-232 port for use with a modem-based dialup connection, a 10/100 Ethernet port, a Gigabit or 10 Gigabit port using copper or fiber, a serial port, a parallel port, or other existing or future ports. Communication port 860 may be chosen depending on a network, such as a Local Area Network (LAN), Wide Area Network (WAN), or any network to which computer system connects. Memory 830 can be Random Access Memory (RAM), or any other dynamic storage device commonly known in the art. Read-only memory 840 can be any static storage device(s) e.g., but not limited to, a Programmable Read Only Memory (PROM) chips for storing static information e.g., start-up or BIOS instructions for processor 870. Mass storage 870 may be any current or future mass storage solution, which can be used to store information and/or instructions. Exemplary mass storage solutions include, but are not limited to, Parallel Advanced Technology Attachment (PATA) or Serial Advanced Technology Attachment (SATA) hard disk drives or solid-state drives (internal or external, e.g., having Universal Serial Bus (USB) and/or Firewire interfaces), e.g. those available from Seagate (e.g., the Seagate Barracuda 7102 family) or Hitachi (e.g., the Hitachi Deskstar 7K1000), one or more optical discs, Redundant Array of Independent Disks (RAID) storage, e.g. an array of disks (e.g., SATA arrays), available from various vendors including Dot Hill Systems Corp., LaCie, Nexsan Technologies, Inc. and Enhance Technology, Inc.

Bus 820 communicatively couples processor(s) 870 with the other memory, storage, and communication blocks. Bus 820 can be, e.g., a Peripheral Component Interconnect (PCI)/PCI Extended (PCI-X) bus, Small Computer System Interface (SCSI), USB, or the like, for connecting expansion cards, drives, and other subsystems as well as other buses, such a front side bus (FSB), which connects processor 870 to a software system.

Optionally, operator and administrative interfaces, e.g., a display, keyboard, joystick, and a cursor control device, may also be coupled to bus 820 to support direct operator interaction with a computer system. Other operator and administrative interfaces can be provided through network connections connected through communication port 860. The external storage device 810 can be any kind of external hard-drives, floppy drives, IOMEGA® Zip Drives, Compact Disc-Read Only Memory (CD-ROM), Compact Disc-ReWritable (CD-RW), Digital Video Disk-Read Only Memory (DVD-ROM). Components described above are meant only to exemplify various possibilities. In no way should the aforementioned exemplary computer system limit the scope of the present disclosure.

Thus, the present disclosure provides a unique and inventive solution for a diagnostics-as-a-service that may include a medical knowledge that has been curated by the medical experts keeping the Indian demography in mind. This will have great applicability in rendering immediate online diagnostic solutions to the people, even to the remotest areas of the Indian populace for assisting medical students to learn differential diagnosis. The system shall benefit healthcare to less privileged patients by collectively increasing the efficiency of conversation with a virtual doctor by asking appropriate questions which help in providing quick diagnostics and reduce overall costs of disease diagnoses, serve as a reference to medical students assisting them in carrying out a triage with the patient to pose the right questions at right time and diagnose the diseases accurately and collect consistent and adequate information from the patient which potentially leads to an accurate disease diagnosis and various machine learning models to learn on correct information

While considerable emphasis has been placed herein on the preferred embodiments, it will be appreciated that many embodiments can be made and that many changes can be made in the preferred embodiments without departing from the principles of the invention. These and other changes in the preferred embodiments of the invention will be apparent to those skilled in the art from the disclosure herein, whereby it is to be distinctly understood that the foregoing descriptive matter is to be implemented merely as illustrative of the invention and not as a limitation.

### Advantages of the Present Disclosure

The present disclosure provides a system and a method for generating adaptive medical queries to determine disease symptoms of a user.

The present disclosure provides a system and a method for facilitating predicting pertinent questions based on the various states as well as a sufficient number of questions with a view to converging a triage.

The present disclosure ensures the enforcement and priority of policy questions as mandated by experts.

The present disclosure provides a system and a method for enabling dependency between successive questions based on the answers to all previous questions.

The present disclosure provides a system and a method for covering questions based on contexts like age, gender, prior diseases, risk factors, lab tests, body organs, and body systems of the user.

The present disclosure provides benefits to healthcare to less privileged patients by collectively increasing the efficiency of conversation with a virtual doctor by asking appropriate questions which help in providing quick diagnostics and reducing overall costs of disease diagnoses.

The present disclosure provides a system and a method for serving as a reference to medical students assisting them in carrying out a triage with the patient to pose the right questions at right time and diagnose the diseases accurately.

The present disclosure provides a system and a method for collecting consistent and adequate information from the patient which potentially leads to an accurate disease diagnosis and various machine learning models to learn the correct information.

### RESERVATION OF RIGHTS

A portion of the disclosure of this patent document contains material which is subject to intellectual property rights such as, but are not limited to, copyright, design, trademark, IC layout design, and/or trade dress protection, belonging to Jio Platforms Limited (JPL) or its affiliates (hereinafter referred as owner). The owner has no objection to the facsimile reproduction by anyone of the patent document or the patent disclosure, as it appears in the Patent and Trademark Office patent files or records, but otherwise reserves all rights whatsoever. All rights to such intellectual property are fully reserved by the owner.

We claim:

1. A system (110) for generating adaptive medical queries to determine disease symptoms, the system (110) comprising:
a processor (202);
a memory (204) coupled to the processor (202), wherein the memory (204) comprises processor-executable instructions, which on execution, cause the processor (202) to:
receive one or more inputs from a user (102), in response to one or more queries corresponding to one or more symptoms associated with a disease of the user (102);
determine, if a previous query from the one or more queries is a symptomatic query, and determine, if each of the one or more inputs from the user (102) is an affirmative input;
extract a set of contextual attributes corresponding to a medical context, from the one or more inputs received from the user (102), when the one or more queries is the symptomatic query, and each of the one or more inputs from the user (102) is the affirmative input;
identify batch-wise candidate attributes from the extracted set of contextual attributes, and sort the batch-wise candidate attributes corresponding to each of the one or more symptoms, in a display order;
map the one or more symptoms in the sorted batch-wise candidate attributes to the disease, by searching a medical knowledge database;
calculate a disease-symptom weighed score for the mapped one or more symptoms to the disease, by retrieving a symptom bucket corresponding to the disease in a knowledge graph;
filter the one or more symptoms based on an age and a gender of the user (102), and sort the one or more symptoms based on the calculated disease-symptom weighed score and an inter-dependency on other attributes in the batch-wise candidate attributes of each symptom;
transmit the filtered and sorted one or more symptoms to a natural language generation layer for converting the one or more symptoms, attribute canonical names, and unique Identities (IDs) to a human-understandable form; and
generate subsequently, one or more adaptive medical queries in the human-understandable form, by predicting the one or more adaptive medical queries based on the converted one or more symptoms, the attribute canonical names, and the unique Identities (IDs), to determine the disease of the user (102).

2. The system (110) as claimed in claim 1, wherein, when the determined previous query from the one or more queries is not the symptomatic query, and the determined each of the one or more inputs from the user (102) is not the affirmative input, the processor (202) is further configured to:
receive one or more symptoms of the disease as an input to one or more queries which are reasoning-based queries, from the user (102);
determine top ‘n’ disease scores with a highest probability, based on the received one or more symptoms of the disease, and the age and the gender of the user (102);
determine top ‘k’ disease scores, by calculating a heuristic score using the received one or more symptoms;
determine candidate symptoms of the determine top ‘k’ disease scores, and compute candidate symptom scores based on the symptom bucket, and pre-defined disease;
analyze, if the top ‘n’ disease scores are greater or equal to the top ‘k’ disease scores with respect to the computed candidate symptom scores;
map the candidate symptoms to the disease, by searching the medical knowledge database, when the top ‘n’ disease scores are greater or equal to the top ‘k’ disease scores;
calculate the disease-symptom weighed score for the mapped candidate symptoms to the disease, by retrieving a symptom bucket corresponding to the disease in a knowledge graph;
filter the one or more symptoms based on the age and the gender of the user (102), and sort the one or more symptoms based on the calculated disease-symptom weighed score and the pre-defined deceased scores; and
transmit the filtered and sorted candidate symptoms to the natural language generation layer for converting the candidate symptoms to the human-understandable form.

3. The system (110) as claimed in claim 1, wherein, for calculating the disease-symptom weighed score, the processor (202) is configured to:
iterate over all diseases, to sum the disease-symptom weighted score for each disease;
determine a probability score of each disease, for the summed-up disease-symptom weighted score;
determine the sum of the probability score of all the diseases;
calculate a normalized disease score from the probability score of each disease and the sum of the probability score of all the diseases;
calculate a symptom score of each disease, based on the disease-symptom weightage score and the normalized disease score; and
update the symptom score for each disease, and update a final symptom score, by summing the symptom score of each disease.

4. The system (110) as claimed in claim 1, wherein the one or more adaptive medical queries are predicted by analyzing previous inputs from the user (102), based on:
previous inputs corresponding to the demographic of the user (102);
previous inputs corresponding to the symptoms of the user (102); and
previous inputs corresponding to the attributes.

5. The system (110) as claimed in claim 1, wherein the processor (202) is further configured to:
determine at least one of an insurance policy, an insurance premium, a health index, and a preventive measures guided by one or more health authorities, upon receiving a response from the user for the one or more adaptive medical queries; and
generate the determined at least one of the insurance policy, the insurance premium, the health index, and the preventive measures guided by one or more health authorities.

6. The system (110) as claimed in claim 1, wherein the medical context comprises at least one of attributes scores of the user (102), lifestyles of the user (102), demographics of the user (102), medical histories of the user (102), family histories of the user (102), prior diseases of the user (102), risk factors of the user (102), lab-tests of the user (102), problems in body organs of the user (102), and body nature of the user (102).

7. The system (110) as claimed in claim 1, wherein the one or more inputs comprise at least one of personal information of the user (102), demographic information comprising the age, the gender of the user (102).

8. The system (110) as claimed in claim 1, wherein the generated one or more adaptive medical queries in the human-understandable form are rule-based queries.

9. A method for generating adaptive medical queries to determine disease symptoms, the method comprising:
receiving, by a processor (202) associated with a system (110), one or more inputs from a user (102), in response to one or more queries corresponding to one or more symptoms associated with a disease of the user (102);
determining, by the processor (202), if a previous query from the one or more queries is a symptomatic query, and determining, if each of the one or more inputs from the user (102) is an affirmative input;
extracting, by the processor (202), a set of contextual attributes corresponding to a medical context, from the one or more inputs received from the user (102), when the one or more queries is the symptomatic query, and each of the one or more inputs from the user (102) is the affirmative input;
identifying, by the processor (202), batch-wise candidate attributes from the extracted set of contextual attributes, and sorting the batch-wise candidate attributes corresponding to each of the one or more symptoms, in a display order;
mapping, by the processor (202), the one or more symptoms in the sorted batch-wise candidate attributes to the disease, by searching a medical knowledge database;
calculating, by the processor (202), a disease-symptom weighed score for the mapped one or more symptoms to the disease, by retrieving a symptom bucket corresponding to the disease in a knowledge graph;
filtering, by the processor (202), the one or more symptoms based on an age and a gender of the user (102), and sorting the one or more symptoms based on the calculated disease-symptom weighed score and an inter-dependency on other attributes in the batch-wise candidate attributes of each symptom;
transmitting, by the processor (202), the filtered and sorted one or more symptoms to a natural language generation layer for converting the one or more symptoms, attribute canonical names, and unique Identities (IDs) to a human-understandable form; and
generating, by the processor (202), subsequently, one or more adaptive medical queries in the human-understandable form, by predicting the one or more adaptive medical queries based on the converted one or more symptoms, the attribute canonical names, and the unique Identities (IDs), to determine the disease of the user (102).

10. The method as claimed in claim 9, wherein, when the determined previous query from the one or more queries is not the symptomatic query, and the determined each of the one or more inputs from the user (102) is not the affirmative input, the method further comprises:
receiving, by the processor (202), one or more symptoms of the disease as an input to one or more queries which are reasoning-based queries, from the user (102);
determining, by the processor (202), top ‘n’ disease scores with a highest probability, based on the received one or more symptoms of the disease, and the age and the gender of the user (102);
determining, by the processor (202), top ‘k’ disease scores, by calculating a heuristic score using the received one or more symptoms;
determining, by the processor (202), candidate symptoms of the determined top ‘k’ disease scores, and computing candidate symptom scores based on the symptom bucket, and a pre-defined disease scores;
analyzing, by the processor (202), if the top ‘n’ disease scores are greater or equal to the top ‘k’ disease scores with respect to the computed candidate symptom scores;
mapping, by the processor (202), the candidate symptoms to the disease, by searching the medical knowledge database, when the top ‘n’ disease scores are greater or equal to the top ‘k’ disease scores;
calculating, by the processor (202), the disease-symptom weighed score for the mapped candidate symptoms to the disease, by retrieving a symptom bucket corresponding to the disease in a knowledge graph;
filtering, by the processor (202), the one or more symptoms based on the age and the gender of the user (102), and sorting the one or more symptoms based on the calculated disease-symptom weighed score and the pre-defined deceased scores; and
transmitting, by the processor (202), the filtered and sorted candidate symptoms to the natural language generation layer for converting the candidate symptoms to the human-understandable form.

11. The method as claimed in claim 9, wherein the medical context comprises at least one of attributes of the user (102), lifestyles of the user (102), demographics of the user (102), medical histories of the user (102), family histories of the user (102), prior diseases of the user (102), risk factors of the user (102), lab-tests of the user (102), problems in body organs of the user (102), and body nature of the user (102).

12. The method as claimed in claim 9, wherein the one or more inputs comprise at least one of personal information of the user (102), demographic information comprising the age, the gender of the user (102).

13. The method as claimed in claim 9, wherein the generated one or more adaptive medical queries in the human-understandable form are rule-based queries.

14. The method as claimed in claim 9, wherein the one or more adaptive medical queries are predicted by analyzing previous inputs from the user (102), based on:
previous inputs corresponding to the demographic of the user (102);
previous inputs corresponding to the symptoms of the user (102); and
previous inputs corresponding to the attributes.

15. The method as claimed in claim 9 further comprises:
determining, by the processor (202), at least one of an insurance policy, an insurance premium, a health index, and a preventive measures guided by one or more health authorities, upon receiving a response from the user for the one or more adaptive medical queries; and
generating, by the processor (202), the determined at least one of the insurance policy, the insurance premium, the health index, and the preventive measures guided by one or more health authorities.

16. The method as claimed in claim 9, wherein calculating the disease-symptom weighed score further comprises:
iterating, by the processor (202), over all diseases, to sum the disease-symptom weighted score for each disease;
determining, by the processor (202), a probability score of each disease, for the summed-up disease-symptom weighted score;
determining, by the processor (202), the sum of the probability score of all the diseases;
calculating, by the processor (202), a normalized disease score from the probability score of each disease and the sum of the probability score of all the diseases;
calculating, by the processor (202), a symptom score of each disease, based on the disease-symptom weightage score and the normalized disease score; and
updating, by the processor (202), the symptom score for each disease, and updating a final symptom score, by summing the symptom score of each disease.

17. A User Equipment (UE) for generating adaptive medical queries to determine disease symptoms, the UE comprising:
a processor (202);
a memory (204) coupled to the processor (202), wherein the memory (204) comprises processor-executable instructions, which on execution, cause the processor (202) to:
receive one or more inputs from a user (102), in response to one or more queries corresponding to one or more symptoms associated with a disease of the user (102);
determine, if a previous query from the one or more queries is a symptomatic query, and determine, if each of the one or more inputs from the user (102) is an affirmative input;
extract a set of contextual attributes corresponding to a medical context, from the one or more inputs received from the user (102), when the one or more queries is the symptomatic query, and each of the one or more inputs from the user (102) is the affirmative input;
identify batch-wise candidate attributes from the extracted set of contextual attributes, and sort the batch-wise candidate attributes corresponding to each of the one or more symptoms, in a display order;
map the one or more symptoms in the sorted batch-wise candidate attributes to the disease, by searching a medical knowledge database;
calculate a disease-symptom weighed score for the mapped one or more symptoms to the disease, by retrieving a symptom bucket corresponding to the disease in a knowledge graph;
filter the one or more symptoms based on an age and a gender of the user (102), and sort the one or more symptoms based on the calculated disease-symptom weighed score and an inter-dependency on other attributes in the batch-wise candidate attributes of each symptom;
transmit the filtered and sorted one or more symptoms to a natural language generation layer for converting the one or more symptoms, attribute canonical names, and unique Identities (IDs) to a human-understandable form; and
generate subsequently, one or more adaptive medical queries in the human-understandable form, by predicting the one or more adaptive medical queries based on the converted one or more symptoms, the attribute canonical names, and the unique Identities (IDs), to determine the disease of the user (102).
